(19) United States
(12) Patent Application Publication
(10) Pub. No.: US 2026/0191521 A1
(43) Pub. Date: Jul. 9, 2026

(54) IMPLANTER CAPABLE OF SIMULTANEOUSLY IMPLANTING BONE ANCHOR, PATCH AND SUTURE

(71) Applicant: THE SECOND AFFILIATED HOSPITAL ZHEJIANG UNIVERSITY SCHOOL OF MEDICINE, Hangzhou (CN)

(72) Inventor: Weiliang SHEN, Hangzhou (CN)

(21) Appl. No.: 19/132,812

(22) PCT Filed: Feb. 5, 2024
(86) PCT No.: PCT/CN2024/075883

§ 371 Date: May 25, 2025

(30) Foreign Application Priority Data
Aug. 9, 2023 (CN) 202310998491.2

(51) Int. Cl. A61B 17/04

(52) U.S. Cl. CPC A61B 17/0401; A61B 2017/0409; A61B 2017/0464

(57) ABSTRACT

An implanter enables simultaneous delivery of a bone anchor, a patch, and sutures is provided. The device comprises a driving rod having a proximal drive end configured to releasably engage the head of the bone anchor, the rod carrying a stepped winding segment around which the patch is wrapped, and a cannula telescopically mounted over the driving rod with a distal end arranged to bear against the anchor head. The patch is pre-secured to the anchor by a first suture and carries a second suture for attachment to tendon or other soft tissue. In use, the assembly is advanced in a single step, the anchor is fixed to bone, and the patch is subsequently tied to soft tissue with the second suture, eliminating a separate patch-fixation step and thereby shortening surgical time.

### CROSS-REFERENCE TO RELATED APPLICATION

This application is a national stage application of International Patent Application No. PCT/CN2024/075883, filed on Feb. 5, 2024, which claims the priority of Chinese Patent Application No. 2023109984912 entitled “IMPLANTER CAPABLE OF SIMULTANEOUSLY IMPLANTING BONE ANCHOR, PATCH AND SUTURE” filed with the Chinese Patent Office on Aug. 9, 2023, which is incorporated herein by reference in its entirety.

### TECHNICAL FIELD

The present disclosure relates to the technical field of medical devices, and in particular to an implanter capable of simultaneously implanting bone anchor, patch and suture.

### BACKGROUND

Rotator cuff tear is a common shoulder joint disease, mainly involving the injury of tendon soft tissue in shoulder joint caused by exercise, with the incidence rate increasing gradually with age. Rotator cuff tear is a common cause of the pain of shoulder joint, reduced mobility and diminished function, accounting for about 60% of shoulder lesions. Arthroscopic rotator cuff repair with patch has become one of the most effective treatments for rotator cuff tears with poor conservative treatment. The rotator cuff patch is mainly placed at a site with large-area rotator cuff injury following the repair of conventional surgical repair, and plays a role as a scaffold, that is, cell proliferation, tissue reunion and tendon-bone interface repair are promoted through cell recruitment and cell adhesion, thus inducing and promoting the rapid healing of the site with rotator cuff tissue injury after rotator cuff repair.

The conventional arthroscopic rotator cuff repair with the patch includes the following steps: firstly, fixing the bone anchor with a suture to the bone tissue by a tool, then fixing the suture to the soft tissue, finally implanting the patch by another tool, and then fixedly connecting the patch to the bone tissue and soft tissue. The surgery requires a variety of implantation tools, which makes the surgery procedures cumbersome and time-consuming, and will increase the possibility of surgical infection and other risks.

Therefore, how to provide an implanter which can simplify the surgery procedures, save the surgery time, ensure the anchoring effect of a bone anchor, and implant a bone anchor, a patch and a suture simultaneously is an urgent problem for those skilled in the art.

### SUMMARY

In view of this, the present disclosure provides an implanter which can simplify the surgery procedures, save the surgery time, ensure the anchoring effect of a bone anchor, and implant a bone anchor, a patch and a suture simultaneously.

To achieve the objective above, the present disclosure employs the following technical solution:
a driving rod, where one end of the driving rod is a driving end that is adapted to insert into a head end of a bone anchor, and a rod wall of the driving rod is provided with a stepwise winding shaft section for winding the patch in a flat manner; and
a sleeve, where the sleeve is sleeved on the driving rod, and one end of the sleeve can be abutted against a head end surface of the bone anchor.

The patch is connected to the bone anchor by a first suture, and the patch is further provided with a second suture for connecting tendon soft tissue.

As can be known from the above the technical solution, compared with the prior art, the present disclosure provides an implanter capable of simultaneously implanting a bone anchor, a patch and a suture simultaneously. During use, the patch and the bone anchor are pre-connected by the first suture, then the bone anchor is inserted into the driving end and the patch is wound on the stepwise winding shaft section in a flat manner, and then the sleeve is moved to the driving end to cover the patch, thus preventing the patch from being exposed, and preventing the patch from being polluted by external impurities during the surgery. Moreover, the patch is limited to the driving rod by the sleeve, thus preventing the patch from separating from the driving rod. In this case, one end of the sleeve is abutted against the head end of the bone anchor, and then after the patch and the bone anchor enter the human body, the other end of the driving rod is rotated to screw and the bone anchor is fixed to the bone tissue. Afterwards, the sleeve is lifted upwards to expose the patch, and then the driving rod is pulled outwards, making the patch separated from the driving rod. It should be noted that the above actions are completed in the human body. In this process, on the one hand, the patch is connected to the bone anchor by the first suture, and the patch is disengaged from the driving rod as the bone anchor is anchored in the bone and the driving rod is removed. On the other hand, as the bone anchor is still abutted against by the sleeve, the problem that the bone anchor is taken out from the bone tissue when the driving rod is withdrawn is effectively prevented, thus ensuring the anchoring effect of the bone anchor. Finally, the patch is deployed, and the patch is stitched on the soft tissue by the second suture, so as to play a role in promoting the healing of the bone tissue and the soft tissue.

Therefore, the patch, the bone anchor and the suture can be implanted into a human body simultaneously by the implanter, then the bone anchor is fixed to bone hard tissue, and finally, the patch is fixed to the soft tissue by the second suture. The step of implanting and fixing the patch is omitted. The bone anchor, the patch and the suture can be implanted by implanting the driving rod once. The surgery procedures are simplified, and the surgery time of the doctor can be saved.

Further, the other end of the sleeve is provided with a lock button, and a lock part of the lock button is in contact and locked with the rod wall of the driving rod. A first clamping groove and a second clamping groove are formed in the rod wall of the driving rod and are separate to each other, and the lock part of the lock button is clamped in the first clamping groove or the second clamping groove.

The beneficial effects generated by adopting the above technical solution is as follows: when the lock part of the lock button is clamped in the first clamping groove, the sleeve is located at the top of the driving rod, at this time, the patch can be wound on the stepwise winding shaft section. When the lock part of the lock button is clamped in the second clamping groove, the sleeve is located at the bottom of the driving rod, and at this time, the sleeve covers the outside of the patch, preventing the patch from being exposed from the driving rod. Therefore, the driving rod is provided with the first clamping groove and the second clamping groove, and with the cooperation of the lock button, fixed positions for upward and downward movement of the sleeve can be achieved, which is easy for the doctor to operate quickly during the surgery.

Further, the other end of the sleeve is provided with a button housing, one sidewall of the button housing is provided with a button hole, and the lock button includes:
a lock pressing block, where the lock pressing block is provided with a sleeve hole sleeved on the driving rod, the lock pressing block is telescopically arranged in the button hole, and a hole edge of the sleeve hole is the lock part, which is clamped onto a groove wall of the first clamping groove or the second clamping groove, and one side end, extending out of the button hole, of the lock pressing block is a pressing end; and
a spring, where the spring is arranged in the button hole, and has one end abutted against the bottom of the button hole and the other end abutted against the other side end, opposite to the pressing end, of the lock pressing block, so as to push the lock pressing block to move, and thus enabling the hole edge to be clamped onto the groove wall of the first clamping groove or the second clamping groove.

The beneficial effects generated by adopting the above technical solution is as follows: when the sleeve needs to be moved, by pressing the pressing end of the lock pressing block, the hole edge of the sleeve hole on the lock pressing block is disengaged from the first clamping groove or the second clamping groove, at this time, the spring is compressed, and then the sleeve can be moved. When the sleeve moves to a position of the first clamping groove or the second clamping groove, under the action of the spring, the hole edge of the sleeve hole on the lock pressing block is clamped in the first clamping groove or the second clamping groove again, thus achieving the fixation of the sleeve and the driving rod. The lock button is simple in structure and easy for the doctor to operate.

Further, each of the first clamping groove and the second clamping groove is an annular groove structure.

The beneficial effects generated by adopting the above technical solution is as follows: even when the sleeve rotates, the lock part of the lock button can be clamped and locked in either the first clamping groove or the second clamping groove.

Further, an operation handle is fixedly connected to the other end of the driving rod.

The beneficial effects generated by adopting the above technical solution is as follows: it is easy for the doctor to operate the driving rod.

Further, the rod wall of the driving rod is provided with a placement groove for accommodating the first suture and the second suture in a length direction of the driving rod.

The beneficial effects generated by adopting the above technical solution is as follows: the first suture and the second suture can be concealed along the driving rod, making the driving end in fit with the bone anchor better, which can prevent the driving rod from being affected by the winding of a scattered suture when screwing the driving rod.

Further, the patch is triangular, a rectangular, or a composite shape combining a rectangle and a triangle.

The beneficial effects generated by adopting the above technical solution is as follows: the patch employs a triangle, a rectangle, a composite shape combining both the triangle and the rectangle, which is conducive to laying the patch flat in situ.

Further, when the first suture is an adjustable loop, the adjustable loop is formed by winding one suture, and includes an adjustable ring and two tensioning ends a and b for adjusting the size of the adjustable ring.

Further, two ends of the first suture are the tensioning end a and the tensioning end b, respectively. The tensioning end a is wrapped around a circle to form a first coil part, and then the tensioning end a penetrates through a first collar on the first suture from a point a1 of the first collar and exits from a point a2 of the first collar, the first collar is at a side of the tensioning end a, and the tensioning end b passes through the first coil part and is wrapped around another circle to form a second coil part, and then the tensioning end b penetrates through a second collar on the first suture line from a point b1 of the second collar and exits from a point b2 of the second collar, the second collar is at a side of the tensioning end b; the first suture between the point a2 of the first collar and the point b2 of the second collar is a connecting part connected to the bone anchor, the patch is connected to an interlocking part of the first coil part and the second coil part, and the connecting part, the first coil part and the second coil part enclose to form the adjustable ring.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a structural diagram of an implanter for a bone anchor according to the present disclosure;

FIG. 2 is a schematic diagram of an exploded structure of a driving rod and a bone anchor;

FIG. 3 is an enlarged structural diagram of part A in FIG. 2;

FIG. 4 is an enlarged structural diagram of part B in FIG. 2;

FIG. 5 is a structural diagram of a lock button when locked;

FIG. 6 is an expanded view of a triangular patch and a bone anchor after being implanted in human body;

FIG. 7 is an expanded view of a rectangular patch and a bone anchor after being implanted in human body;

FIG. 8 is an expanded view of a patch composed of a triangle and a rectangle and a bone anchor after being implanted in human body;

FIG. 9 is a diagram of a triangular patch and a bone anchor connected by an adjustable suture;

FIG. 10 is a diagram of a rectangular patch and a bone anchor connected to each other by an adjustable suture;

FIG. 11 is a diagram of a patch composed of a triangle and a rectangle and a bone anchor connected to each other by an adjustable suture;

FIG. 12 is a structural diagram of an adjustable loop;

FIG. 13 is a diagram showing a connection between an adjustable loop and a bone anchor as well as a patch.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

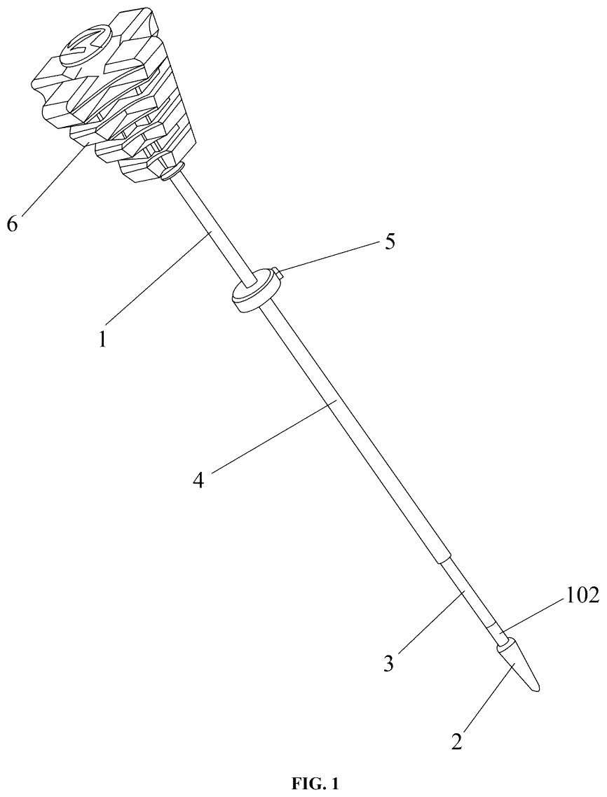

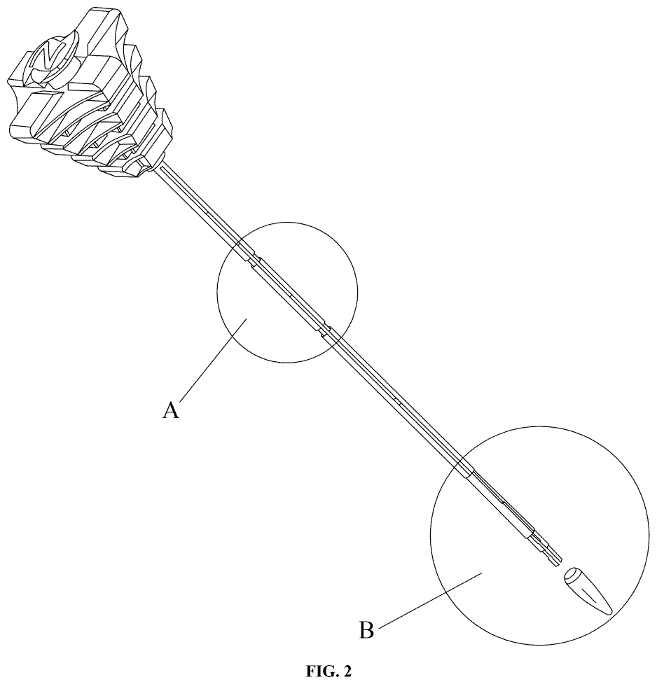

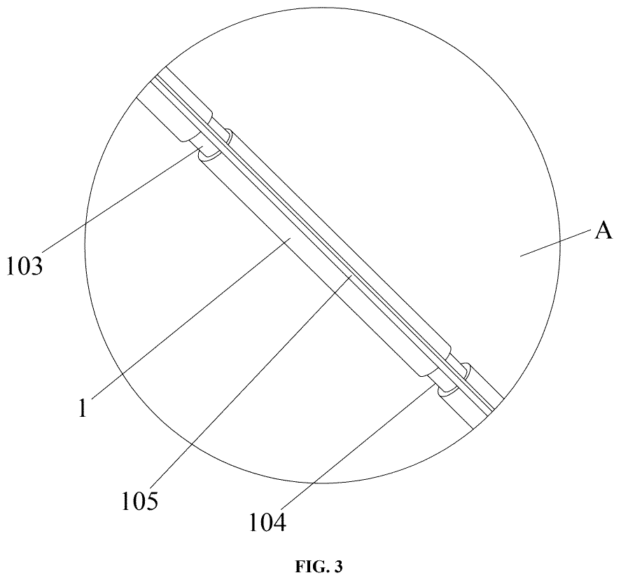

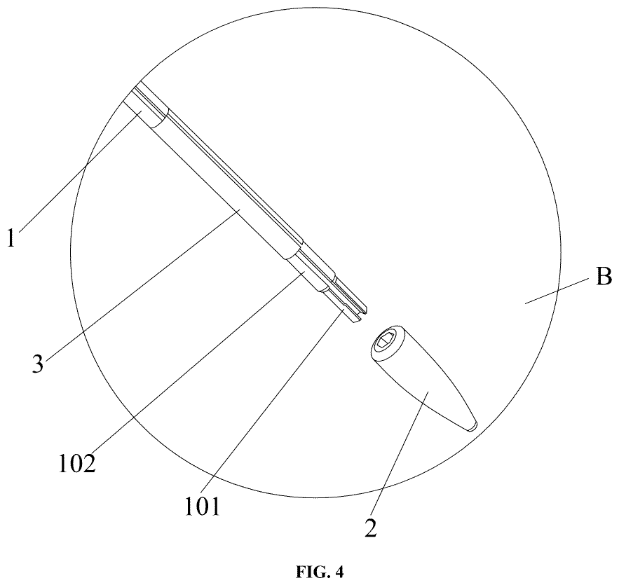

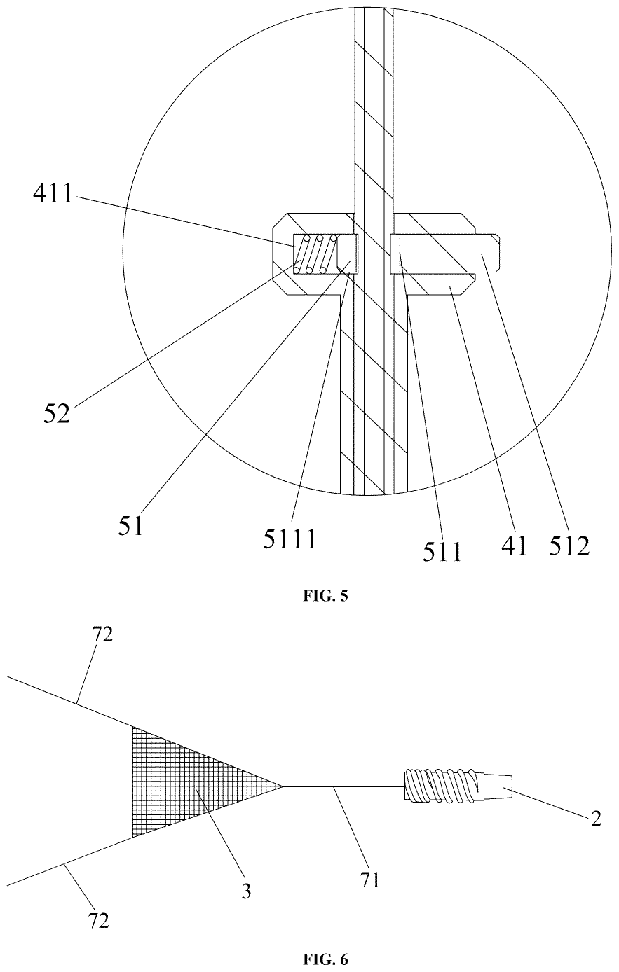

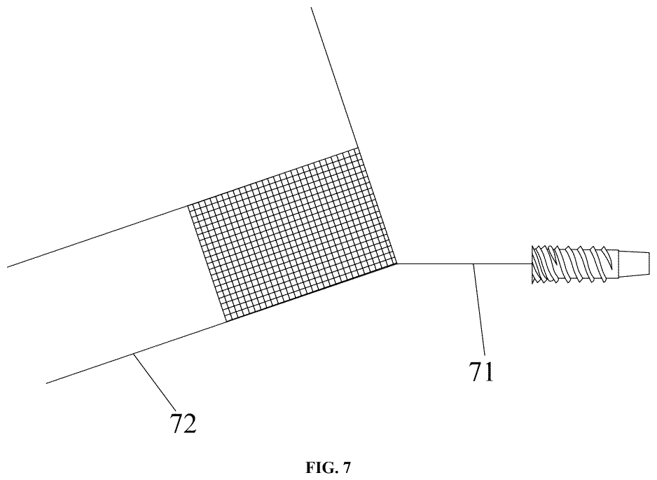

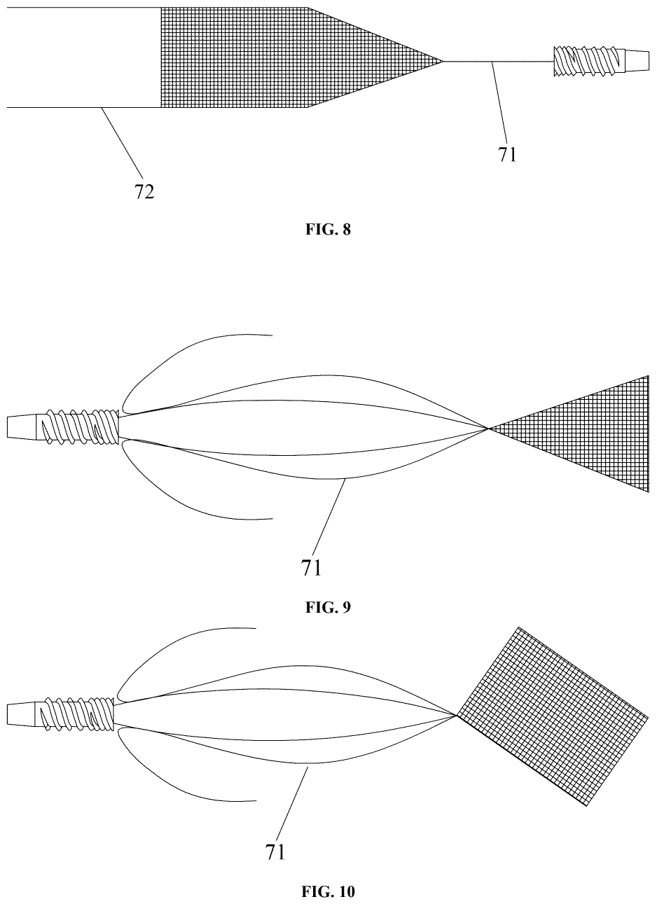

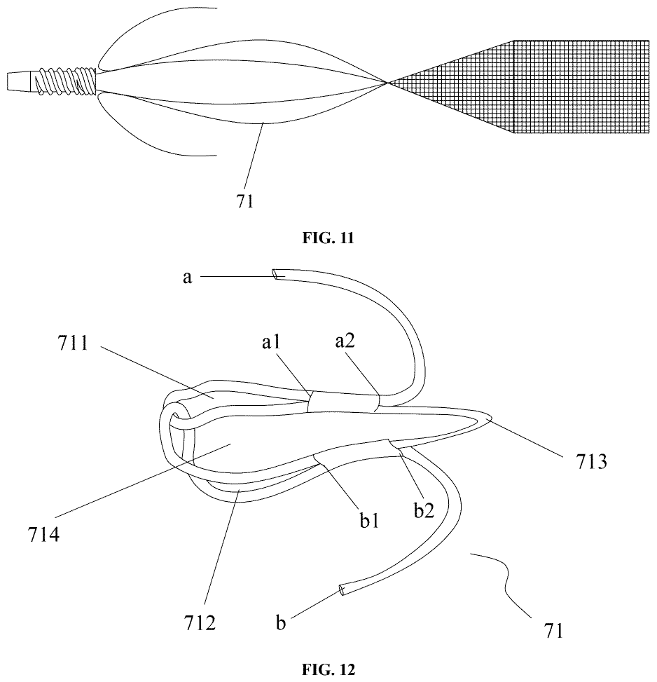

As shown in FIG. 1 to FIG. 13, an embodiment of the present disclosure provides an implanter capable of simultaneously implanting a bone anchor, a patch and a suture simultaneously. In this embodiment, a structure of the implanter includes:
a driving rod 1, where one end of the driving rod 1 is a driving end 101 that is adapted to insert into a head end of a bone anchor 2. Specifically, a head end of the bone anchor is provided with a square hole, the driving end is a square rod inserted into the square hole, and a rod wall of the driving rod 1 is provided with a stepwise winding shaft section 102 for winding the patch 3 in a flat manner; and
a sleeve 4, where the sleeve 4 is sleeved on the driving rod 1, and one end of the sleeve 4 can be abutted against the head end surface of the bone anchor 2; and
the patch 3 is connected to the bone anchor 2 by a first suture 71, and the patch 3 is further provided with a second suture 72 for connecting tendon soft tissue.

The other end of the sleeve 4 is provided with a lock button 5, and a lock part of the lock button 5 is in contact and locked with the rod wall of the driving rod 1. A first clamping groove 103 and a second clamping groove 104 are formed in the rod wall of the driving rod 1 and are separate to each other, and a lock part of the lock button 5 is clamped in either the first clamping groove 103 or the second clamping groove 104.

A button housing 41 is fixed to the other end of the sleeve 4, and one sidewall of the button housing 41 is provided with a button hole 411. The lock button 5 includes:
a lock pressing block 51, where the lock pressing block 51 is provided with a sleeve hole 511 sleeved outside the driving rod 1, the lock pressing block 51 is telescopically arranged in the button hole 411, and a hole edge 5111 of the sleeve hole 511 is the lock part, which is clamped onto a groove wall of the first clamping groove 103 or the second clamping groove 104, and one side end, extending out of the button hole 411, of the lock pressing block 51 is a pressing end 512;
a spring 52, where the spring 52 is arranged in the button hole 411, and has one end abutted against the bottom of the button hole 411 and the other end abutted against the other side end, opposite to the pressing end 512, of the lock pressing block 51, so as to push the lock pressing block 51 to move, and thus enabling the hole edge 5111 to be clamped onto the groove wall of either the first clamping groove 103 or the second clamping groove 104.

Each of the first clamping groove 103 and the second clamping groove 104 is an annular groove structure.

An operation handle 6 is fixedly connected to the other end of the driving rod 1.

In an embodiment, the rod wall of the driving rod 1 is provided with a placement groove 105 for accommodating the first suture 71 and the second suture 72 in a length direction of the driving rod 1.

In the embodiment, the patch 3 is triangular or rectangular, or a composite shape combining a rectangle and a triangle.

In the embodiment, the first suture 71 is a suture with a fixed length, or an adjustable loop with an adjustable length. The adjustable loop is a suture with an adjustable length. When the first suture is the adjustable loop, the adjustable loop is formed by winding one suture, and includes an adjustable ring and two tensioning ends a and b for adjusting the size of the adjustable ring. The suture with the adjustable length is the suture used by existing medical staff, with the adjustable length. Therefore, a connection distance between the bone anchor and the patch can be adjusted to better meet the surgical requirements.

A winding method of the adjustable loop is as follows: two ends of the first suture 71 are the tensioning end a and the tensioning end b, respectively. The tensioning end a is wrapped around a circle to form a first coil part 711, and then the tensioning end a penetrates a first collar on the first suture 71 from a point a1 of the first collar and exits from a point a2 of the first collar, the first collar is at a side of the tensioning end a; the tensioning end b passes through the first coil part 711 and is wrapped around another circle to form a second coil part 712, and then the tensioning end b penetrates through a second collar on the first suture line 71 from a point b1 of the second collar and exits from a point b2 of the second collar, the second collar is at a side of the tensioning end b. The first suture 71 between the point a2 of the first collar and the point b2 of the second collar is a connecting part 713 connected to the bone anchor 2, the patch 3 is connected to an interlocking part of the first coil part 711 and the second coil part 712, and the connecting part 713, the first coil part 711 and the second coil part 712 enclose to form the adjustable ring 714. During use, the tensioning end a and the tensioning end b are dragged to make the first coil part and the second coil part become smaller, which makes the adjustable ring become smaller, thus reducing the distance between the patch and the bone anchor.

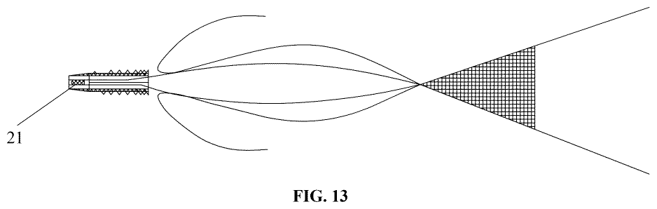

As shown in FIG. 13, a specific connection way of the adjustable loop and the bone anchor and the patch is as follows: the bone anchor 2 is internally provided with a cross beam 21 for hanging the adjustable loop. Specifically, a connecting part of the adjustable loop is hung on the cross beam of the bone anchor, and the patch is connected at the interlocking part of the first coil part and the second coil part. During use, the tensioning end a and the tensioning end b are dragged to make the coils of the first coil part and the second coil part become smaller, which makes the adjustable ring become smaller, thus adjusting the distance between the patch and the bone anchor.

When the patient with small-scale injury of tendon soft tissue is repaired, a specific operation process of implanting the bone anchor is as follows: the patch and the bone anchor are pre-connected by the first suture, then the bone anchor is inserted into the driving end and the patch is wound on the stepwise winding shaft section in a flat manner, and then the sleeve is moved to the driving end to cover the patch, and the lock button is used to lock and fix the sleeve and the driving rod, thus preventing the patch from being exposed, and preventing the patch from being polluted by external impurities during the surgery. Moreover, the patch is limited to the driving rod by the sleeve, thus preventing the patch from disengaging from the driving rod. In this case, one end of the sleeve is abutted against the head end of the bone anchor, and then after the patch and the bone anchor enter the human body, the other end of the driving rod is rotated to screw and the bone anchor is fixed to the bone tissue, the lock button is pressed to make the driving rod unlocked from the sleeve, and then the driving rod can be pulled outwards. In this process, on the one hand, the patch is connected to the bone anchor by the first suture, and the patch is disengaged from the driving rod as the bone anchor is anchored in the bone and the driving rod is removed. On the other hand, as the bone anchor is still abutted against by the sleeve, the problem that the bone anchor is taken out from the bone tissue when the driving rod is withdrawn is effectively prevented, thus ensuring the anchoring effect of the bone anchor. Finally, the patch is deployed, and the patch is stitched on the soft tissue by the second suture, so as to play a role in promoting the healing of the bone tissue and the soft tissue.

The implanter enables simultaneous implantation of the patch, the bone anchor and the suture integrated as a whole, then the bone anchor is fixed to bone hard tissue, and finally, the patch is fixed to the soft tissue by the second suture. The step of implanting and fixing the patch is omitted, the bone anchor. The patch and the suture can be implanted by implanting the driving rod once. The surgery procedures are simplified, and the surgery time of the doctor can be saved.

The implanter is not only suitable for small-scale local injury of tendon soft tissue, but also can employ multiple patches and bone anchors to repair large-scale injured tissue at the same time.

Various embodiments in this specification are described in a progressive way, and each embodiment focuses on the difference from other embodiments, the same and similar parts between the various embodiments can be referred to each other. Since the device disclosed by the embodiments corresponds to the method disclosed by the embodiments, the description is relatively simple, and the reference is made to the descriptions in the method for related parts.

According to the above description of the disclosed embodiments, those skilled in the art can implement or practice the present disclosure. Various modifications made to these embodiments are apparent to those skilled in the art, the general principle defined herein may be implemented in other embodiments without departing from the spirit and scope of the present disclosure. Hence, the present disclosure is not limited to these embodiments disclosed herein, but is to conform to the widest scope in accordance with the principles and novel features disclosed herein.

1. An implanter capable of simultaneously implanting a bone anchor, a patch and a suture, comprising:
a driving rod (1), wherein one end of the driving rod (1) is a driving end (101), the driving end (101) is adapted to insert into a head end of a bone anchor (2), and a rod wall of the driving rod (1) is provided with a stepwise winding shaft section (102) for winding the patch (3) in a flat manner; and
a sleeve (4), wherein the sleeve (4) is sleeved on the driving rod (1), and one end of the sleeve (4) is able to be abutted against a head end surface of the bone anchor (2); and
the patch (3) is connected to the bone anchor (2) by a first suture (71), and the patch (3) is further provided with a second suture (72) for connecting tendon soft tissue.

2. The implanter capable of simultaneously implanting a bone anchor, a patch and a suture simultaneously according to claim 1, wherein an other end of the sleeve (4) is provided with a lock button (5); a first clamping groove (103) and a second clamping groove (104) are formed in the rod wall of the driving rod (1) and are separate to each other, and a lock part of the lock button (5) is clamped in the first clamping groove (103) or the second clamping groove (104).

3. The implanter capable of simultaneously implanting a bone anchor, a patch and a suture simultaneously according to claim 2, wherein the other end of the sleeve (4) is provided with a button housing (41), one sidewall of the button housing (41) is provided with a button hole (411), and the lock button (5) comprises:
a lock pressing block (51), wherein the lock pressing block (51) is provided with a sleeve hole (511) sleeved on the driving rod (1), the lock pressing block (51) is telescopically arranged in the button hole (411), and a hole edge (5111) of the sleeve hole (511) is the lock part, the hole edge (5111) is clamped onto a groove wall of the first clamping groove (103) or the second clamping groove (104), and one side end, extending out of the button hole (411), of the lock pressing block (51) is a pressing end (512);
a spring (52), wherein the spring (52) is arranged in the button hole (411), and has one end abutted against a bottom of the button hole (411) and an other end abutted against an other side end, opposite to the pressing end (512), of the lock pressing block (51), so as to push the lock pressing block (51) to move, and thus enabling the hole edge (5111) to be clamped onto the groove wall of the first clamping groove (103) or the second clamping groove (104).

4. The implanter capable of simultaneously implanting a bone anchor, a patch and a suture simultaneously according to claim 1, wherein an operation handle (6) is fixedly connected to an other end of the driving rod (1).

5. The implanter capable of simultaneously implanting a bone anchor, a patch and a suture simultaneously according to claim 1, wherein the rod wall of the driving rod (1) is provided with a placement groove (105) for accommodating the first suture (71) and the second suture (72) in a length direction of the driving rod (1).

6. The implanter capable of simultaneously implanting a bone anchor, a patch and a suture simultaneously according to claim 1, wherein the patch (3) is triangular, rectangular, or a composite shape combining a rectangle and a triangle.

7. The implanter capable of simultaneously implanting a bone anchor, a patch and a suture simultaneously according to claim 1, wherein the first suture (71) is a suture with a fixed length, or an adjustable loop with an adjustable length.

8. The implanter capable of simultaneously implanting a bone anchor, a patch and a suture simultaneously according to claim 7, wherein when the first suture (71) is the adjustable loop, the adjustable loop is formed by winding one suture, and comprises an adjustable ring (714) and two tensioning ends a and b for adjusting a size of the adjustable ring (714).

9. The implanter capable of simultaneously implanting a bone anchor, a patch and a suture simultaneously according to claim 8, wherein two ends of the first suture (71) are the tensioning end a and the tensioning end b, respectively; the tensioning end a is wrapped around a circle to form a first coil part (711), and then the tensioning end a penetrates through a first collar on the first suture (71) from a point a1 of the first collar and exits from a point a2 of the first collar, the first collar is at a side of the tensioning end a, and the tensioning end b passes through the first coil part (711) and is wrapped around another circle to form a second coil part (712), and then the tensioning end b penetrates through a second collar on the first suture line (71) from a point b1 of the second collar and exits from a point b2 of the second collar, the second collar is at a side of the tensioning end b; the first suture (71) between the point a2 of the first collar and the point b2 of the second collar is a connecting part (713) connected to the bone anchor (2), the patch (3) is connected to an interlocking part of the first coil part (711) and the second coil part (712), and the connecting part (713), the first coil part (711) and the second coil part (712) enclose to form the adjustable ring (714).

10. The implanter capable of simultaneously implanting a bone anchor, a patch and a suture simultaneously according to claim 2, wherein an operation handle (6) is fixedly connected to an other end of the driving rod (1).

11. The implanter capable of simultaneously implanting a bone anchor, a patch and a suture simultaneously according to claim 3, wherein an operation handle (6) is fixedly connected to an other end of the driving rod (1).

12. The implanter capable of simultaneously implanting a bone anchor, a patch and a suture simultaneously according to claim 2, wherein the rod wall of the driving rod (1) is provided with a placement groove (105) for accommodating the first suture (71) and the second suture (72) in a length direction of the driving rod (1).

13. The implanter capable of simultaneously implanting a bone anchor, a patch and a suture simultaneously according to claim 3, wherein the rod wall of the driving rod (1) is provided with a placement groove (105) for accommodating the first suture (71) and the second suture (72) in a length direction of the driving rod (1).

14. The implanter capable of simultaneously implanting a bone anchor, a patch and a suture simultaneously according to claim 2, wherein the patch (3) is triangular, rectangular, or a composite shape combining a rectangle and a triangle.

15. The implanter capable of simultaneously implanting a bone anchor, a patch and a suture simultaneously according to claim 3, wherein the patch (3) is triangular, rectangular, or a composite shape combining a rectangle and a triangle.

16. The implanter capable of simultaneously implanting a bone anchor, a patch and a suture simultaneously according to claim 2, wherein the first suture (71) is a suture with a fixed length, or an adjustable loop with an adjustable length.

17. The implanter capable of simultaneously implanting a bone anchor, a patch and a suture simultaneously according to claim 3, wherein the first suture (71) is a suture with a fixed length, or an adjustable loop with an adjustable length.
